(19) United States
(12) Patent Application Publication
(10) Pub. No.: US 2004/0162286 A1
(43) Pub. Date: Aug. 19, 2004

(54) Substituted 6-(2-tolyl)-triazolopyrimidines as fungicides

(76) Inventors: Jordi Tormo i Blasco, Limburgerhof (DE); Hubert Sauter, Mannheim (DE); Bernd Muller, Frankenthal (DE); Markus Gewehr, Kastellaun (DE); Wassilios Grammenos, Ludwigshafen (DE); Thomas Grote, Wachenheim (DE); Andreas Gypser, Mannheim (DE); Joachim Rheinheim, Ludwigshafen (DE); Ingo Rose, Mannheim (DE); Peter Schafer, Ottersheim (DE); Frank Schieweck, Hessheim (DE); Michael Rack, Heidelberg (DE); Eberhard Ammermann, Heppenheim (DE); Siegfried Strathmann, Limburgerhof (DE); Gisela Lorenz, Hambach (DE); Reinhard Stierl, Mutterstadt (DE)

Correspondence Address: KEIL & WEINKAUF, 1350 CONNECTICUT AVENUE, N.W., WASHINGTON, DC, 20036, US

(21) Appl. No.: 10/483,600

(22) PCT Filed: Jul. 8, 2002
(86) PCT No.: PCT/EP02/07578

(30) Foreign Application Priority Data
Jul. 18, 2001 (EP) 01117402.6

(51) Int. Cl. A61K 31/541; A61K 31/5377; A61K 31/519; C07D 487/04

(52) U.S. Cl. 514/227.8; 514/234.5; 514/259.31; 544/60; 544/118; 544/263

(57) ABSTRACT

Substituted 6-(2-tolyl)-triazolopyrimidines of formula (I) in which R¹ and R² independently denote hydrogen or alkyl, alkenyl, alkynyl, or alkadienyl, haloalkyl, haloalkenyl, cycloalkyl, phenyl, naphthyl, or 5- or 6-membered heterocyclyl, containing one to four nitrogen atoms or one to three nitrogen atoms and one sulfur or oxygen atom, or 5- or 6-membered heteroaryl, containing one to four nitrogen atoms or one to three nitrogen atoms and one sulfur or oxygen atom, or where R¹ and R² radicals may be unsubstituted or substituted as defined in the description, or R¹ and R² together with the interjacent nitrogen atom represent a 5- or 6-membered heterocyclic ring, containing one to four nitrogen atoms or one to three nitrogen atoms and one sulfur or oxygen atom, which may be substituted; R³ is halogen, cyano, alkyl, alkoxy, haloalkyl, or C(═O)A, wherein A is hydrogen, hydroxy, alkyl, amino, or mono- or dialkyl-amino; n is an integer from 1 to 4; and X is halogen, cyano, alkyl, alkoxy, haloalkoxy or alkenyloxy; processes for their preparation, compositions containing them and to their use for combating phytopathogenic fungi.

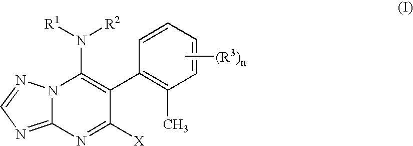

The invention relates to substituted 6-(2-tolyl)-triazolopyrimidines of formula I

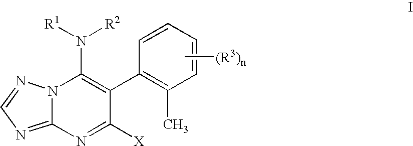

in which

R¹ and R² independently denote hydrogen or C₁-C₁₀-alkyl, C₂-C₁₀-alkenyl, C₂-C₁₀-alkynyl, or C₄-C₁₀-alkadienyl, C₁-C₁₀-haloalkyl, C₂-C₁₀-haloalkenyl, C₃-C₁₀-cycloalkyl, phenyl, naphthyl, or

5- or 6-membered heterocyclyl, containing one to four nitrogen atoms or one to three nitrogen atoms and one sulfur or oxygen atom, or

5- or 6-membered heteroaryl, containing one to four nitrogen atoms or one to three nitrogen atoms and one sulfur or oxygen atom, or

where R¹ and R² radicals may be unsubstituted or partly or fully halogenated or may carry one to three groups R^{a},

R^{a} is cyano, nitro, hydroxyl, C₁-C₆-alkyl, C₃-C₆-cycloalkyl, C₁-C₆-alkoxy, C₁-C₆-alkylthio, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and C₁-C₄-alkylenedioxy; or

R¹ and R² together with the interjacent nitrogen atom represent a 5- or 6-membered heterocyclic ring, containing one to four nitrogen atoms or one to three nitrogen atoms and one sulfur or oxygen atom, which may be substituted by one to three R^{a} radicals;

R³ is halogen, cyano, C₁-C₁₀-alkyl, C₁-C₁₀-alkoxy, C₁-C₁₀-haloalkyl, or C(═O)A, wherein

A is hydrogen, hydroxy, C₁-C₈-alkyl, C₁-C₈-alkoxy, amino, C₁-C₈-alkylamino, or di-(C₁-C₈-alkyl)-amino;

n is an integer from 1 to 4; and

X is halogen, cyano, C₁-C₆-alkyl, C₁-C₆-alkoxy, C₁-C₆-haloalkoxy or C₃-C₈-alkenyloxy.

Moreover, the invention relates to processes for their preparation, compositions containing them and to their use for combating phytopathogenic fungi.

6-Phenyl-7-amino-triazolopyrimidines are generally known from U.S. Pat. No. 4,567,262 and U.S. Pat. No. 5,593,996.

Triazolopyrimidines with a trifluorophenyl group in 6-position are disclosed in WO-A 98/46607 and EP-A 945 453.

From WO-A 98/46608 diverse 6-phenyl-triazolopyrimidines are known, which are substituted in the 7-position by fluorinated alkylamines.

The compounds disclosed in the documents discussed above are said to be active against various phytopathogenic fungi.

It is an object of the present invention to provide compounds having improved fungicidal activity.

We have found that this object is achieved by the compounds defined at the outset. Furthermore, we have found processes for their preparation, compositions comprising them and methods for controlling phytopathogenic fungi using the compounds I.

The compounds of the formula I differ from the compounds known from closest prior art WO-A 98/46608 in the 2-tolyl group, which is further substituted.

The present invention further provides a process for the preparation of compounds of formula I as defined above which comprises reacting 5-amino-triazole with 2-(2-tolyl)-substituted malonic acid ester of formula II, in which

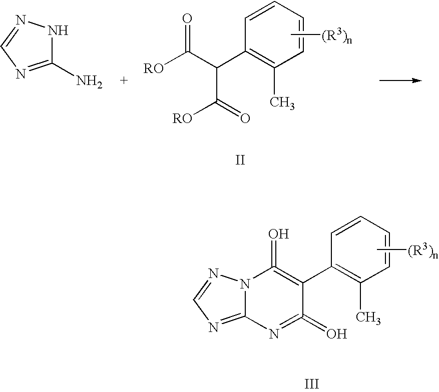

R represents alkyl, preferably C₁-C₆-alkyl, in particular methyl or ethyl, under alkaline conditions, preferably using high boiling tertiary amines as for example tri-n-butylamine as disclosed for example by EP-A 770 615 to yield compounds of formula III.

The resulting 5,7-dihydroxy-6-phenyl-triazolopyrimidine of formula III, wherein R³ and n are as defined for formula I, is subsequently treated with a halogenating agent, preferably with a brominating or chlorinating agent, such as phosphorus oxybromide or phosphorus oxychloride, neat or in the presence of a solvent to give IV.

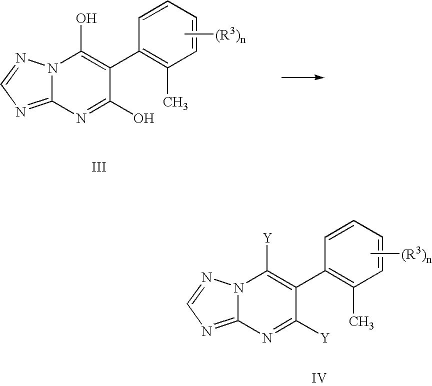

The reaction is suitably carried out at a temperature in the range from 0° C. to 150° C., the preferred reaction temperature being from 80° C. to 125° C. as disclosed for example by EP-A 770 615.

Dihalotriazolopyrimidine IV is further reacted with an amine of formula V

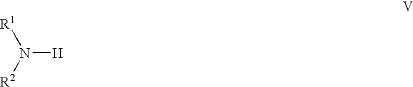

in which R¹ and R² are as defined in formula I to produce compounds of formula I in which X is halogen.

The reaction between the 5,7-dihalo compound IV and the amine of formula V can be carried out under conditions known from WO-A 98/46608. The reaction is preferably carried out in the presence of a solvent. Suitable solvents include ethers, such as dioxane, diethyl ether and, especially, tetrahydrofuran, halogenated hydrocarbons such as dichloromethane and aromatic hydrocarbons, for example toluene.

The reaction is suitably carried out at a temperature in the range from 0° C. to 70° C., the preferred reaction temperature being from 10° C. to 35° C.

It is also preferred that the reaction is carried out in the presence of a base. Suitable bases include tertiary amines, such as triethylamine, and inorganic bases, such as potassium carbonate or sodium carbonate. Alternatively, an excess of the compound of formula V may serve as a base.

Compounds of formula I in which X denotes cyano, C₁-C₆-alkoxy, C₁-C₆-haloalkoxy or C₃-C₈-alkenyloxy can be prepared by reacting compounds I in which X is halogen, preferably chloro, with compounds of formula VI, which are, dependent from the value of X′ to be introduced to yield formula I compounds, an anorganic cyano salt, an alkoxylate, haloalkoxylate or an alkenyloxylate, respectively, preferably in the presence of a a solvent. The cation M in formula VI has minor influence; for practical and economical reasons usually ammonium-, tetraalkylammonium- or alkalimetal- and earth metal salts are preferred.

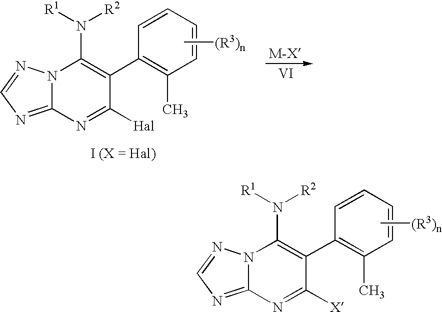

The reaction is suitably carried out at a temperature in the range from 0 to 120° C., the preferred reaction temperature being from 10 to 40° C. [cf. J. Heterocycl. Chem., Vol.12, p. 861-863 (1975)].

Suitable solvents include ethers, such as dioxane, diethyl ether and, especially, tetrahydrofuran, halogenated hydrocarbons such as dichloromethane and aromatic hydrocarbons, for example toluene.

Compounds of formula I in which X denotes C₁-C₆-alkyl can be prepared by reacting compounds I in which X is halogen, preferably chloro, with malonic acid esters of formula VII, wherein X″ denotes H or C₁-C₅-alkyl and R denotes C₁-C₄-alkyl, to compounds of formula VIII and decarboxylation under conditions described in U.S. Pat. No. 5,994,360.

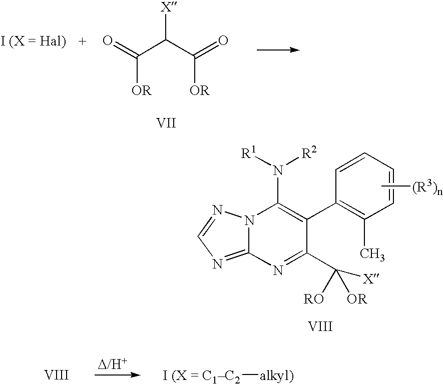

Accordingly, the invention relates to the novel intermediates of formulae II, III and IV.

The compounds of formula II are preferably prepared by reaction of the corresponding substituted bromobenzenes with sodium dialkylmalonates in the presence of a copper(I) salt [cf. Chemistry Letters, pp. 367-370, 1981; EP-A 10 02 788].

The compounds of formula II may also be prepared by reaction of an alkyl 2-(2-tolyl)-acetate with dialkylcarbonate in the presence of a strong base, preferably sodium ethoxide and sodium hydride (cf. Heterocycles, pp. 1031-1047, 1996).

The substituted phenylacetates which are the starting compounds for compounds of formula II are known and commercially available, and/or they are obtainable by generally known methods.

The reaction mixtures are worked up in a customary manner, for example by mixing with water, phase separation and, if required, chromatographic purification of the crude products. Some of the end products are obtained in the form of colorless or slightly brownish, viscous oils, which are purified or freed from volatile components under reduced pressure and at moderately elevated temperatures. If the end products are obtained as solids, purification can also be carried out by recrystallization or digestion.

If individual compounds I are not obtainable by the routes described above, they can be prepared by derivatization of other compounds I.

In the symbol definitions given in the formulae above, collective terms were used which generally represent the following substituents:

halogen: fluorine, chlorine, bromine and iodine;

C₁-C₁₀-alkyl: saturated, straight-chain or branched hydrocarbon radicals having 1 to 10, especially 1 to 6 carbon atoms, for example C₁-C₄-alkyl as mentioned above or pentyl, 1-methylbutyl, 2-methylbutyl, 3-methylbutyl, 2,2-di-methylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

C₂-C₁₀-alkenyl: unsaturated, straight-chain or branched hydrocarbon radicals having 2 to 10, especially 2 to 6 carbon atoms and a double bond in any position, for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl and 2-methyl-2-propenyl; C₂-C₁₀-alkynyl: straight-chain or branched hydrocarbon radicals having 2 to 10, especially 2 to 4 carbon atoms and a triple bond in any position, for example ethynyl, 1-propynyl, 2-propynyl, 1-butynyl, 2-butynyl, 3-butynyl and 1-methyl-2-propynyl;

C₁-C₁₀-haloalkyl and haloalkyl moieties of C₁-C₆-haloalkoxy: straight-chain or branched alkyl groups having 1 to 6 or 10, preferably 1 to 4 carbon atoms (as mentioned above), where the hydrogen atoms in these groups may be partially or fully replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy;

C₃-C₁₀-cycloalkyl: mono- or bicyclic cycloalkyl groups having 3 to 10 carbon atoms; monocyclic groups preferably have 3 to 8, especially 3 to 6 ring members, bicyclic groups preferably have 8 to 10 ring members.

A 5- or 6-membered heterocyclyl group, containing one to four nitrogen atoms or one to three nitrogen atoms and one sulfur or oxygen atom, preferably one oxygen atom, for example 1-pyrimidinyl, 2-pyrimidinyl, morpholin-4-yl.

5-membered heteroaryl, containing one to four nitrogen atoms or one to three nitrogen atoms and one sulfur or oxygen atom:

5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;

6-membered heteroaryl, containing one to four nitrogen atoms:

6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

With respect to their intended use, preference is given to triazolopyrimidines of the formula I having the following substituents, where the preference is valid in each case on its own or in combination:

A preferred cycloalkyl moiety is cyclopentyl being optionally substituted by one or more nitro, cyano, C₁-C₆-alkyl, C₁-C₆-alkoxy groups.

A preferred heteroaryl moiety is pyridyl, pyrimidyl, pyrazolyl or thienyl.

Preference is given to compounds of formula I in which any alkyl or haloalkyl part of the groups R¹ or R², which may be straight chained or branched, contains up to 10 carbon atoms, preferably 1 to 9 carbon atoms, more preferably 2 to 6 carbon atoms, any alkenyl or alkynyl part of the substituents R¹ or R² contains up to 10 carbon atoms, preferably 2 to 9 carbon atoms, more preferably 3 to 6 carbon atoms, any cycloalkyl part of the substituents R¹ or R² contains from 3 to 10 carbon atoms, preferably from 3 to 8 carbon atoms, more preferably from 3 to 6 carbon atoms, and any bicycloalkyl part of the substituents R¹ or R² contains from 5 to 9 carbon atoms, preferably from 7 to 9 carbon atoms. Any alkyl, alkenyl or alkynyl group may be linear or branched.

Likewise, preference is given to compounds of formula I wherein R¹ is not hydrogen.

Compounds of formula I are preferred in which R¹ represents a straight-chained or branched C₁-C₁₀-alkyl, in particular a branched C₃-C₁₀-alkyl group, a C₃-C₈-cycloalkyl, a C₅-C₉-bicycloalkyl, a C₃-C₈-cycloalkyl-C₁-C₆-alkyl, C₁-C₁₀-alkoxy-C₁-C₆-alkyl, a C₁-C₁₀-haloalkyl or a phenyl group being optionally substituted by one to three halogen atoms or C₁-C₁₀-alkyl or C₁-C₁₀-alkoxy groups.

Particular preference is given to compounds I in which R² represents hydrogen, C₁-C₁₀-alkyl or C₁-C₁₀-haloalkyl, in particular hydrogen.

Besides, particular preference is given to compounds I in which R² is hydrogen.

Moreover, particular preference is given to compounds I in which R² is methyl.

Furthermore, particular preference is given to compounds I in which R² is ethyl.

If R¹ denotes C₁-C₁₀-haloalkyl, preferably polyfluorinated alkyl, in particular 2,2,2-trifluoroethyl, 2-(1,1,1-trifluoropropyl) or 2-(1,1,1-trifluorobutyl), R² preferably represents hydrogen.

If R¹ denotes optionally substituted C₃-C₈-cycloalkyl, preferably cyclopentyl or cyclohexyl, R² preferably represents hydrogen or C₁-C₆-alkyl.

Moreover, particular preference is given to compounds I in which R¹ and R² together with the interjacent nitrogen atom form an optionally substituted heterocyclic ring, preferably an optionally substituted C₃-C₇-heterocyclic ring, in particular pyrrolidine, piperidine, tetrahydropyridine, in particular 1,2,3,6-tetrahydropyridine or azepane which is optionally substituted by one or more C₁-C₁₀-alkyl groups.

Preference is given to compounds of formula I in which any alkyl part of the groups R¹ or R², which may be straight chained or branched, contains 1 to 9 carbon atoms, more preferably 2 to 6 carbon atoms, any alkenyl or alkynyl part of the substituents R¹ or R² contains 2 to 9 carbon atoms, more preferably 3 to 6 carbon atoms, any cycloalkyl part of the substituents R¹ or R² contains from 3 to 10 carbon atoms, preferably from 3 to 8 carbon atoms, more preferably from 3 to 6 carbon atoms, and any bicycloalkyl part of the substituents R¹ or R² contains from 7 to 9 carbon atoms. Any alkyl, alkenyl or alkynyl moiety may be linear or branched.

Compounds of formula I are preferred in which R¹ represents a straight-chained or branched C₁-C₁₀-alkyl, in particular branched C₃-C₁₀-alkyl, a C₃-C₈-cycloalkyl, C₅-C₉-bicycloalkyl, C₃-C₈-cycloalkyl-C₁-C₆-alkyl, C₁-C₁₀-alkoxy-C₁-C₆-alkyl, or phenyl being optionally substituted by one to three C₁-C₁₀-alkyl or C₁-C₁₀-alkoxy groups.

Particular preference is given to compounds I in which R² represents hydrogen or C₁-C₁₀-alkyl, in particular hydrogen.

Moreover, particular preference is given to compounds I in which R² is methyl or ethyl.

If R¹ denotes an optionally substituted C₃-C₈-cycloalkyl group, preferably cyclopentyl or cyclohexyl, R² preferably represents hydrogen or C₁-C₆-alkyl.

Moreover, particular preference is given to compounds I in which R¹ and R² together with the interjacent nitrogen atom form an optionally substituted heterocyclic ring, preferably an optionally substituted C₃-C₇-heterocyclic ring, in particular a pyrrolidine, piperidine, tetrahydropyridine, in particular 1,2,3,6-tetrahydropyridine or azepane ring which is optionally substituted by one or more C₁-C₁₀-alkyl groups.

Likewise, particular preference is given to compounds I in which R² is hydrogen.

Particular preference is also given to compounds I in which n has the value 2 and R³ groups are in 4- and 6-position.

Moreover, preference is given to compounds I in which a R³ group is in para-portion.

Furthermore, particular preference is given to compounds I in which (R³)ₙ is 4,6-dimethyl.

Likewise, particular preference is given to compounds I in which (R³)ₙ is 4-(C₁-C₈-alkoxy)carbonyl, especially 4-methoxycarbonyl.

Besides, particular preference is given to compounds I in which (R³)ₙ is 4-methoxy-6-methyl, or 4-fluoro-6-methyl.

Particular preference is also given to compounds I in which (R³)ₙ is 4-fluoro, 6-fluoro, or 4-chloro.

Besides, particular preference is given to compounds I in which X is chloro or bromo, especially chloro.

Moreover, preference is given to compounds I in which X is cyano or methyl.

Furthermore, particular preference is given to compounds I in which X is methoxy, ethoxy, n-propoxy, iso-propoxy, allyloxy, or 3-methylallyloxy.

The particularly preferred embodiments of the intermediates with respect to the variables correspond to those of the radicals X, R¹, R² and R³ of formula I.

Included in the scope of the present invention are (R) and (S) isomers of compounds of general formula I having a chiral center and the racemates thereof, and salts, N-oxides and acid addition compounds.

With respect to their use, particular preference is given to the compounds I compiled in the tables below. The groups mentioned in the tables for a substituent are furthermore for their part, independently of the combination in which they are mentioned, a particularly preferred embodiment of the respective substituents.

Table 1

Compounds of formula I, in which X is chloro, (R³)ₙ is 4-chloro and R¹ and R² correspond to one row in Table A

Table 2

Compounds of formula I, in which X is chloro, (R³)ₙ is 5-chloro and R¹ and R² correspond to one row in Table A

Table 3

Compounds of formula I, in which X is chloro, (R³)ₙ is 6-chloro and R¹ and R² correspond to one row in Table A

Table 4

Compounds of formula I, in which X is chloro, (R³)ₙ is 3-fluoro and R¹ and R² correspond to one row in Table A

Table 5

Compounds of formula I, in which X is chloro, (R³)ₙ is 4-fluoro and R¹ and R² correspond to one row in Table A

Table 6

Compounds of formula I, in which X is chloro, (R³)ₙ is 5-fluoro and R¹ and R² correspond to one row in Table A

Table 7

Compounds of formula I, in which X is chloro, (R³)ₙ is 6-fluoro and R¹ and R² correspond to one row in Table A

Table 8

Compounds of formula I, in which X is chloro, (R³)ₙ is 3-methyl and R¹ and R² correspond to one row in Table A

Table 9

Compounds of formula I, in which X is chloro, (R³)ₙ is 4-methyl and R¹ and R² correspond to one row in Table A

Table 10

Compounds of formula I, in which X is chloro, (R³)ₙ is 5-methyl and R¹ and R² correspond to one row in Table A

Table 11

Compounds of formula I, in which X is chloro, (R³)ₙ is 6-methyl and R¹ and R² correspond to one row in Table A

Table 12

Compounds of formula I, in which X is chloro, (R³)ₙ is 4-methoxy and R¹ and R² correspond to one row in Table A

Table 13

Compounds of formula I, in which X is chloro, (R³)ₙ is 5-methoxy and R¹ and R² correspond to one row in Table A

Table 14

Compounds of formula I, in which X is chloro, (R³)ₙ is 6-methoxy and R¹ and R² correspond to one row in Table A

Table 15

Compounds of formula I, in which X is chloro, (R³)ₙ is 4-trifluoromethyl and R¹ and R² correspond to one row in Table A

Table 16

Compounds of formula I, in which X is chloro, (R³)ₙ is 5-trifluoromethyl and R¹ and R² correspond to one row in Table A

Table 17

Compounds of formula I, in which X is chloro, (R³)ₙ is 6-trifluoromethyl and R¹ and R² correspond to one row in Table A

Table 18

Compounds of formula I, in which X is chloro, (R³)ₙ is 4,6-dimethyl and R¹ and R² correspond to one row in Table A

Table 19

Compounds of formula I, in which X is chloro, (R³)ₙ is 4-methoxy-6-methyl and R¹ and R² correspond to one row in Table A

Table 20

Compounds of formula I, in which X is chloro, (R³)ₙ is 4-fluoro-6-methyl and R¹ and R² correspond to one row in Table A

Table 21

Compounds of formula I, in which X is chloro, (R³)ₙ is 4-methoxycarbonyl and R¹ and R² correspond to one row in Table A

Table 22

Compounds of formula I, in which X is chloro, (R³)ₙ is 4-cyano and R¹ and R² correspond to one row in Table A

Table 23

Compounds of formula I, in which X is bromo, (R³)ₙ is 4-chloro and R¹ and R² correspond to one row in Table A

Table 24

Compounds of formula I, in which X is bromo, (R³)ₙ is 5-chloro and R¹ and R² correspond to one row in Table A

Table 25

Compounds of formula I, in which X is bromo, (R³)ₙ is 6-chloro and R¹ and R² correspond to one row in Table A

Table 26

Compounds of formula I, in which X is bromo, (R³)ₙ is 3-fluoro and R¹ and R² correspond to one row in Table A

Table 27

Compounds of formula I, in which X is bromo, (R³)ₙ is 4-fluoro and R¹ and R² correspond to one row in Table A

Table 28

Compounds of formula I, in which X is bromo, (R³)ₙ is 5-fluoro and R¹ and R² correspond to one row in Table A

Table 29

Compounds of formula I, in which X is bromo, (R³)ₙ is 6-fluoro and R¹ and R² correspond to one row in Table A

Table 30

Compounds of formula I, in which X is bromo, (R³)ₙ is 3-methyl and R¹ and R² correspond to one row in Table A

Table 31

Compounds of formula I, in which X is bromo, (R³)ₙ is 4-methyl and R¹ and R² correspond to one row in Table A

Table 32

Compounds of formula I, in which X is bromo, (R³)ₙ is 5-methyl and R¹ and R² correspond to one row in Table A

Table 33

Compounds of formula I, in which X is bromo, (R³)ₙ is 6-methyl and R¹ and R² correspond to one row in Table A

Table 34

Compounds of formula I, in which X is bromo, (R³)ₙ is 4-methoxy and R¹ and R² correspond to one row in Table A

Table 35

Compounds of formula I, in which X is bromo, (R³)ₙ is 5-methoxy and R¹ and R² correspond to one row in Table A

Table 36

Compounds of formula I, in which X is bromo, (R³)ₙ is 6-methoxy and R¹ and R² correspond to one row in Table A

Table 37

Compounds of formula I, in which X is bromo, (R³)ₙ is 4-trifluoromethyl and R¹ and R² correspond to one row in Table A

Table 38

Compounds of formula I, in which X is bromo, (R³)ₙ is 5-trifluoromethyl and R¹ and R² correspond to one row in Table A

Table 39

Compounds of formula I, in which X is bromo, (R³)ₙ is 6-trifluoromethyl and R¹ and R² correspond to one row in Table A

Table 40

Compounds of formula I, in which X is bromo, (R³)ₙ is 4,6-dimethyl and R¹ and R² correspond to one row in Table A

Table 41

Compounds of formula I, in which X is bromo, (R³)ₙ is 4-methoxy-6-methyl and R¹ and R² correspond to one row in Table A

Table 42

Compounds of formula I, in which X is bromo, (R³)ₙ is 4-fluoro-6-methyl and R¹ and R² correspond to one row in Table A

Table 43

Compounds of formula I, in which X is bromo, (R³)ₙ is 4-methoxycarbonyl and R¹ and R² correspond to one row in Table A

Table 44

Compounds of formula I, in which X is bromo, (R³)ₙ is 4-cyano and R¹ and R² correspond to one row in Table A

Table 45

Compounds of formula I, in which X is cyano, (R³)ₙ is 4-chloro and R¹ and R² correspond to one row in Table A

Table 46

Compounds of formula I, in which X is cyano, (R³)ₙ is 5-chloro and R¹ and R² correspond to one row in Table A

Table 47

Compounds of formula I, in which X is cyano, (R³)ₙ is 6-chloro and R¹ and R² correspond to one row in Table A

Table 48

Compounds of formula I, in which X is cyano, (R³)ₙ is 3-fluoro and R¹ and R² correspond to one row in Table A

Table 49

Compounds of formula I, in which X is cyano, (R³)ₙ is 4-fluoro and R¹ and R² correspond to one row in Table A

Table 50

Compounds of formula I, in which X is cyano, (R³)ₙ is 5-fluoro and R¹ and R² correspond to one row in Table A

Table 51

Compounds of formula I, in which X is cyano, (R³)ₙ is 6-fluoro and R¹ and R² correspond to one row in Table A

Table 52

Compounds of formula I, in which X is cyano, (R³)ₙ is 3-methyl and R¹ and R² correspond to one row in Table A

Table 53

Compounds of formula I, in which X is cyano, (R³)ₙ is 4-methyl and R¹ and R² correspond to one row in Table A

Table 54

Compounds of formula I, in which X is cyano, (R³)ₙ is 5-methyl and R¹ and R² correspond to one row in Table A

Table 55

Compounds of formula I, in which X is cyano, (R³)ₙ is 6-methyl and R¹ and R² correspond to one row in Table A

Table 56

Compounds of formula I, in which X is cyano, (R³)ₙ is 4-methoxy and R¹ and R² correspond to one row in Table A

Table 57

Compounds of formula I, in which X is cyano, (R³)ₙ is 5-methoxy and R¹ and R² correspond to one row in Table A

Table 58

Compounds of formula I, in which X is cyano, (R³)ₙ is 6-methoxy and R¹ and R² correspond to one row in Table A

Table 59

Compounds of formula I, in which X is cyano, (R³)ₙ is 4-trifluoromethyl and R¹ and R² correspond to one row in Table A

Table 60

Compounds of formula I, in which X is cyano, (R³)ₙ is 5-trifluoromethyl and R¹ and R² correspond to one row in Table A

Table 61

Compounds of formula I, in which X is cyano, (R³)ₙ is 6-trifluoromethyl and R¹ and R² correspond to one row in Table A

Table 62

Compounds of formula I, in which X is cyano, (R³)ₙ is 4,6-dimethyl and R¹ and R² correspond to one row in Table A

Table 63

Compounds of formula I, in which X is cyano, (R³)ₙ is 4-methoxy-6-methyl and R¹ and R² correspond to one row in Table A

Table 64

Compounds of formula I, in which X is cyano, (R³)ₙ is 4-fluoro-6-methyl and R¹ and R² correspond to one row in Table A

Table 65

Compounds of formula I, in which X is cyano, (R³)ₙ is 4-methoxycarbonyl and R¹ and R² correspond to one row in Table A

Table 66

Compounds of formula I, in which X is cyano, (R³)ₙ is 4-cyano and R¹ and R² correspond to one row in Table A

Table 67

Compounds of formula I, in which X is methoxy, (R³)ₙ is 4-chloro and R¹ and R² correspond to one row in Table A

Table 68

Compounds of formula I, in which X is methoxy, (R³)ₙ is 5-chloro and R¹ and R² correspond to one row in Table A

Table 69

Compounds of formula I, in which X is methoxy, (R³)ₙ is 6-chloro and R¹ and R² correspond to one row in Table A

Table 70

Compounds of formula I, in which X is methoxy, (R³)ₙ is 3-fluoro and R¹ and R² correspond to one row in Table A

Table 71

Compounds of formula I, in which X is methoxy, (R³)ₙ is 4-fluoro and R¹ and R² correspond to one row in Table A

Table 72

Compounds of formula I, in which X is methoxy, (R³)ₙ is 5-fluoro and R¹ and R² correspond to one row in Table A

Table 73

Compounds of formula I, in which X is methoxy, (R³)ₙ is 6-fluoro and R¹ and R² correspond to one row in Table A

Table 74

Compounds of formula I, in which X is methoxy, (R³)ₙ is 3-methyl and R¹ and R² correspond to one row in Table A

Table 75

Compounds of formula I, in which X is methoxy, (R³)ₙ is 4-methyl and R¹ and R² correspond to one row in Table A

Table 76

Compounds of formula I, in which X is methoxy, (R³)ₙ is 5-methyl and R¹ and R² correspond to one row in Table A

Table 77

Compounds of formula I, in which X is methoxy, (R³)ₙ is 6-methyl and R¹ and R² correspond to one row in Table A

Table 78

Compounds of formula I, in which X is methoxy, (R³)ₙ is 4-methoxy and R¹ and R² correspond to one row in Table A

Table 79

Compounds of formula I, in which X is methoxy, (R³)ₙ is 5-methoxy and R¹ and R² correspond to one row in Table A

Table 80

Compounds of formula I, in which X is methoxy, (R³)ₙ is 6-methoxy and R¹ and R² correspond to one row in Table A

Table 81

Compounds of formula I, in which X is methoxy, (R³)ₙ is 4-trifluoromethyl and R¹ and R² correspond to one row in Table A

Table 82

Compounds of formula I, in which X is methoxy, (R³)ₙ is 5-trifluoromethyl and R¹ and R² correspond to one row in Table A

Table 83

Compounds of formula I, in which X is methoxy, (R³)ₙ is 6-trifluoromethyl and R¹ and R² correspond to one row in Table A

Table 84

Compounds of formula I, in which X is methoxy, (R³)ₙ is 4,6-dimethyl and R¹ and R² correspond to one row in Table A

Table 85

Compounds of formula I, in which X is methoxy, (R³)ₙ is 4-methoxy-6-methyl and R¹ and R² correspond to one row in Table A

Table 86

Compounds of formula I, in which X is methoxy, (R³)ₙ is 4-fluoro-6-methyl and R¹ and R² correspond to one row in Table A

Table 87

Compounds of formula I, in which X is methoxy, (R³)ₙ is 4-methoxycarbonyl and R¹ and R² correspond to one row in Table A

Table 88

Compounds of formula I, in which X is methoxy, (R³)ₙ is 4-cyano and R¹ and R² correspond to one row in Table A

Table 89

Compounds of formula I, in which X is methyl, (R³)ₙ is 4-chloro and R¹ and R² correspond to one row in Table A

Table 90

Compounds of formula I, in which X is methyl, (R³)ₙ is 5-chloro and R¹ and R² correspond to one row in Table A

Table 91

Compounds of formula I, in which X is methyl, (R³)ₙ is 6-chloro and R¹ and R² correspond to one row in Table A

Table 92

Compounds of formula I, in which X is methyl, (R³)ₙ is 3-fluoro and R¹ and R² correspond to one row in Table A

Table 93

Compounds of formula I, in which X is methyl, (R³)ₙ is 4-fluoro and R¹ and R² correspond to one row in Table A

Table 94

Compounds of formula I, in which X is methyl, (R³)ₙ is 5-fluoro and R¹ and R² correspond to one row in Table A

Table 95

Compounds of formula I, in which X is methyl, (R³)ₙ is 6-fluoro and R¹ and R² correspond to one row in Table A

Table 96

Compounds of formula I, in which X is methyl, (R³)ₙ is 3-methyl and R¹ and R² correspond to one row in Table A

Table 97

Compounds of formula I, in which X is methyl, (R³)ₙ is 4-methyl and R¹ and R² correspond to one row in Table A

Table 98

Compounds of formula I, in which X is methyl, (R³)ₙ is 5-methyl and R¹ and R² correspond to one row in Table A

Table 99

Compounds of formula I, in which X is methyl, (R³)ₙ is 6-methyl and R¹ and R² correspond to one row in Table A

Table 100

Compounds of formula I, in which X is methyl, (R³)ₙ is 4-methoxy and R¹ and R² correspond to one row in Table A

Table 101

Compounds of formula I, in which X is methyl, (R³)ₙ is 5-methoxy and R¹ and R² correspond to one row in Table A

Table 102

Compounds of formula I, in which X is methyl, (R³)ₙ is 6-methoxy and R¹ and R² correspond to one row in Table A

Table 103

Compounds of formula I, in which X is methyl, (R³)ₙ is 4-trifluoromethyl and R¹ and R² correspond to one row in Table A

Table 104

Compounds of formula I, in which X is methyl, (R³)ₙ is 5-trifluoromethyl and R¹ and R² correspond to one row in Table A

Table 105

Compounds of formula I, in which X is methyl, (R³)ₙ is 6-trifluoromethyl and R¹ and R² correspond to one row in Table A

Table 106

Compounds of formula I, in which X is methyl, (R³)ₙ is 4,6-dimethyl and R¹ and R² correspond to one row in Table A

Table 107

Compounds of formula I, in which X is methyl, (R³)ₙ is 4-methoxy-6-methyl and R¹ and R² correspond to one row in Table A

Table 108

Compounds of formula I, in which X is methyl, (R³)ₙ is 4-fluoro-6-methyl and R¹ and R² correspond to one row in Table A

Table 109

Compounds of formula I, in which X is methyl, (R³)ₙ is 4-methoxycarbonyl and R¹ and R² correspond to one row in Table A

Table 110

Compounds of formula I, in which X is methyl, (R³)ₙ is 4-cyano and R¹ and R² correspond to one row in Table A

TABLE A

| | |
|---|---|
| | I |
| 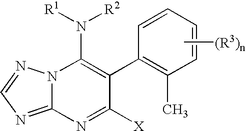 | |

| No. | R¹ | R² |
|---|---|---|
| A-1 | H | H |
| A-2 | CH₂CH₃ | H |
| A-3 | CH₂CH₃ | CH₃ |
| A-4 | CH₂CH₃ | CH₂CH₃ |
| A-5 | CH₂CF₃ | H |
| A-6 | CH₂CF₃ | CH₃ |
| A-7 | CH₂CF₃ | CH₂CH₃ |
| A-8 | CH₂CCl₃ | H |
| A-9 | CH₂CCl₃ | CH₃ |
| A-10 | CH₂CCl₃ | CH₂CH₃ |
| A-11 | CH₂CH₂CH₃ | H |
| A-12 | CH₂CH₂CH₃ | CH₃ |
| A-13 | CH₂CH₂CH₃ | CH₂CH₃ |
| A-14 | CH₂CH₂CH₃ | CH₂CH₂CH₃ |
| A-15 | CH(CH₃)₂ | H |
| A-16 | CH(CH₃)₂ | CH₃ |
| A-17 | CH(CH₃)₂ | CH₂CH₃ |
| A-18 | (±) CH(CH₃)—CH₂CH₃ | H |
| A-19 | (±) CH(CH₃)—CH₂CH₃ | CH₃ |
| A-20 | (±) CH(CH₃)—CH₂CH₃ | CH₂CH₃ |
| A-21 | (S) CH(CH₃)—CH₂CH₃ | H |
| A-22 | (S) CH(CH₃)—CH₂CH₃ | CH₃ |
| A-23 | (S) CH(CH₃)—CH₂CH₃ | CH₂CH₃ |
| A-24 | (R) CH(CH₃)—CH₂CH₃ | H |
| A-25 | (R) CH(CH₃)—CH₂CH₃ | CH₃ |
| A-26 | (R) CH(CH₃)—CH₂CH₃ | CH₂CH₃ |
| A-27 | (±) CH(CH₃)—CH(CH₃)₂ | H |
| A-28 | (±) CH(CH₃)—CH(CH₃)₂ | CH₃ |
| A-29 | (±) CH(CH₃)—CH(CH₃)₂ | CH₂CH₃ |
| A-30 | (S) CH(CH₃)—CH(CH₃)₂ | H |
| A-31 | (S) CH(CH₃)—CH(CH₃)₂ | CH₃ |
| A-32 | (S) CH(CH₃)—CH(CH₃)₂ | CH₂CH₃ |
| A-33 | (R) CH(CH₃)—CH(CH₃)₂ | H |
| A-34 | (R) CH(CH₃)—CH(CH₃)₂ | CH₃ |
| A-35 | (R) CH(CH₃)—CH(CH₃)₂ | CH₂CH₃ |
| A-36 | (±) CH(CH₃)—C(CH₃)₃ | H |
| A-37 | (±) CH(CH₃)—C(CH₃)₃ | CH₃ |
| A-38 | (±) CH(CH₃)—C(CH₃)₃ | CH₂CH₃ |
| A-39 | (S) CH(CH₃)—C(CH₃)₃ | H |
| A-40 | (S) CH(CH₃)—C(CH₃)₃ | CH₃ |
| A-41 | (S) CH(CH₃)—C(CH₃)₃ | CH₂CH₃ |
| A-42 | (R) CH(CH₃)—C(CH₃)₃ | H |
| A-43 | (R) CH(CH₃)—C(CH₃)₃ | CH₃ |
| A-44 | (R) CH(CH₃)—C(CH₃)₃ | CH₂CH₃ |
| A-45 | (±) CH(CH₃)—CF₃ | H |
| A-46 | (±) CH(CH₃)—CF₃ | CH₃ |
| A-47 | (±) CH(CH₃)—CF₃ | CH₂CH₃ |
| A-48 | (S) CH(CH₃)—CF₃ | H |
| A-49 | (S) CH(CH₃)—CF₃ | CH₃ |
| A-50 | (S) CH(CH₃)—CF₃ | CH₂CH₃ |
| A-51 | (R) CH(CH₃)—CF₃ | H |
| A-52 | (R) CH(CH₃)—CF₃ | CH₃ |
| A-53 | (R) CH(CH₃)—CF₃ | CH₂CH₃ |
| A-54 | (±) CH(CH₃)—CCl₃ | H |
| A-55 | (±) CH(CH₃)—CCl₃ | CH₃ |
| A-56 | (±) CH(CH₃)—CCl₃ | CH₂CH₃ |
| A-57 | (S) CH(CH₃)—CCl₃ | H |
| A-58 | (S) CH(CH₃)—CCl₃ | CH₃ |
| A-59 | (S) CH(CH₃)—CCl₃ | CH₂CH₃ |
| A-60 | (R) CH(CH₃)—CCl₃ | H |
| A-61 | (R) CH(CH₃)—CCl₃ | CH₃ |
| A-62 | (R) CH(CH₃)—CCl₃ | CH₂CH₃ |
| A-63 | CH₂C(CH₃)═CH₂ | H |
| A-64 | CH₂C(CH₃)═CH₂ | CH₃ |
| A-65 | CH₂C(CH₃)═CH₂ | CH₂CH₃ |
| A-66 | cyclopentyl | H |
| A-67 | cyclopentyl | CH₃ |
| A-68 | cyclopentyl | CH₂CH₃ |
| A-69 | —(CH₂)₂CH(CH₃)(CH₂)_{2—} | |
| A-70 | CH₂CF₂CF₃ | H |
| A-71 | CH₂CF₂CF₃ | CH₃ |
| A-72 | CH₂CF₂CF₃ | CH₂CH₃ |
| A-73 | CH₂CF₂CF₂CF₃ | H |
| A-74 | CH₂CF₂CF₂CF₃ | CH₃ |
| A-75 | CH₂CF₂CF₂CF₃ | CH₂CH₃ |

The compounds I are suitable as fungicides. They have outstanding activity against a broad spectrum of phytopathogenic fungi, in particular from the classes of the Ascomycetes, Deuteromycetes, Phycomycetes and Basidiomycetes. Some of them act systemically, and they can be employed in crop protection as foliar- and soil-acting fungicides.

They are especially important for controlling a large number of fungi on a variety of crop plants such as wheat, rye, barley, oats, rice, maize, grass, bananas, cotton, soya, coffee, sugar cane, grapevines, fruit species, ornamentals and vegetables such as cucumbers, beans, tomatoes, potatoes and cucurbits, and on the seeds of these plants.

Specifically, they are suitable for controlling the following plant diseases:

Alternaria species, Podosphaera species, Sclerotinia species, Physalospora canker on vegetables and fruit,

Botrytis cinerea (gray mold) an strawberries, vegetables, ornamentals and grapevines,

Corynespora cassiicola on cucumbers,

Colletotrichum species on fruit and vegetables,

Diplocarpon rosae on roses,

Elsinoe fawcetti and Diaporthe citri on citrus fruit,

Sphaerotheca species on cucurbits, strawberries and roses,

Cercospora species on peanuts, sugar beets and aubergines,

Erysiphe cichoracearum on cucurbits,

Leveillula taurica on paprika, tomatoes and aubergines,

Mycosphaerella species on apples and japanese apricot,

Phyllactinia kakicola, Gloesporium kaki on japanese apricot,

Gymnosporangium yamadae, Leptothyrium pomi, Podosphaera leucotricha and Gloedes pomigena on apples,

Cladosporium carpophilum on pears and japanese apricot,

Phomopsis species on pears,

Phytophthora species on citrus fruit, potatoes, onions, especially Phytophthora infestans on potatoes and tomatoes,

Blumeria graminis (powdery mildew) on cereals,

Fusarium- and Verticillium species on various plants,

Glomerella cingulata on tee,

Drechslera- and Bipolaris species on cereals and rice,

Mycosphaerella species on bananas and peanuts,

Plasmopara viticola on grapevines,

Personospora species on onions, spinach and chrysantemum,

Phaeoisariopsis vitis and Sphaceloma ampelina on grapefruits,

Pseudocercosporella herpotrichoides on wheat and barley,

Pseudoperonospora species on hop and cucumbers,

Puccinia species and Typhula species on cereals and turf,

Pyricularia oryzae on rice,

Rhizoctonia species on cotton, rice and turf,

Stagonospora nodorum and Septoria tritici on wheat,

Uncinula necator on grapevines,

Ustilago species on cereals and sugar cane, and

Venturia species (scab) on apples and pears.

Moreover, the compounds I are suitable for controlling harmful fungi such as Paecilomyces variotii in the protection of materials (e.g. wood, paper, paint dispersions, fibers and tissues) and in the protection of stored products.

The compounds I are applied by treating the fungi, or the plants, seeds, materials or the soil to be protected against fungal infection, with a fungicidally active amount of the active ingredients. Application can be effected both before and after infection of the materials, plants or seeds by the fungi.

In general, the fungicidal compositions comprise from 0.1 to 95, preferably 0.5 to 90, % by weight of active ingredient.

When used in crop protection, the rates of application are from 0.01 to 2.0 kg of active ingredient per ha, depending on the nature of the effect desired.

In the treatment of seed, amounts of active ingredient of from 0.001 to 0.1 g, preferably 0.01 to 0.05 g, are generally required per kilogram of seed.

When used in the protection of materials or stored products, the rate of application of active ingredient depends on the nature of the field of application and on the effect desired. Rates of application conventionally used in the protection of materials are, for example, from 0.001 g to 2 kg, preferably 0.005 g to 1 kg, of active ingredient per cubic meter of material treated.

The compounds I can be converted into the customary formulations, e.g. solutions, emulsions, suspensions, dusts, powders, pastes and granules. The use form depends on the particular purpose; in any case, it should guarantee a fine and uniform distribution of the compound according to the invention.

The formulations are prepared in a known manner, e.g. by extending the active ingredient with solvents and/or carriers, if desired using emulsifiers and dispersants, it also being possible to use other organic solvents as auxiliary solvents if water is used as the diluent. Auxiliaries which are suitable are essentially: solvents such as aromatics (e.g. xylene), chlorinated aromatics (e.g. chlorobenzenes), paraffins (e.g. mineral oil fractions), alcohols (e.g. methanol, butanol), ketones (e.g. cyclohexanone), amines (e.g. ethanolamine, dimethylformamide) and water; carriers such as ground natural minerals (e.g. kaolins, clays, talc, chalk) and ground synthetic minerals (e.g. highly-disperse silica, silicates); emulsifiers such as non-ionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates and fatty acids and their alkali metal and alkaline earth metal salts, salts of sulfated fatty alcohol glycol ether, condensates of sulfonated naphthalene and naphthalene derivatives with formaldehyde, condensates of naphthalene or of napthalenesulfonic acid with phenol or formaldehyde, polyoxyethylene octylphenyl ether, ethoxylated isooctylphenol, octylphenol, nonylphenol, alkylphenol polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, e.g. benzene, toluene, xylene, paraffin, tetrahydronaphthalene, alkylated naphthalenes or their derivatives, methanol, ethanol, propanol, butanol, chloroform, carbon tetrachloride, cyclohexanol, cyclohexanone, chlorobenzene, isophorone, strongly polar solvents, e.g. dimethylformamide, dimethyl sulfoxide, N-methylpyrrolidone and water.

Powders, materials for scattering and dusts can be prepared by mixing or concomitantly grinding the active substances with a solid carrier.

Granules, e.g. coated granules, impregnated granules and homogeneous granules, can be prepared by binding the active ingredients to solid carriers. Examples of solid carriers are mineral earths, such as silicas, silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, e.g. ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise of from 0.01 to 95% by weight, preferably from 0.1 to 90% by weight, of the active ingredient. The active ingredients are employed in a purity of from 90% to 100%, preferably 95% to 100% (according to NMR spectrum).

The following are exemplary formulations:

I. 5 parts by weight of a compound according to the invention are mixed intimately with 95 parts by weight of finely divided kaolin. This gives a dust which comprises 5% by weight of the active ingredient.

II. 30 parts by weight of a compound according to the invention are mixed intimately with a mixture of 92 parts by weight of pulverulent silica gel and 8 parts by weight of paraffin oil which had been sprayed onto the surface of this silica gel. This gives a formulation of the active ingredient with good adhesion properties (comprises 23% by weight of active ingredient).

III. 10 parts by weight of a compound according to the invention are dissolved in a mixture composed of 90 parts by weight of xylene, 6 parts by weight of the adduct of 8 to 10 mol of ethylene oxide and 1 mol of oleic acid N-monoethanolamide, 2 parts by weight of calcium dodecylbenzenesulfonate and 2 parts by weight of the adduct of 40 mol of ethylene oxide and 1 mol of castor oil (comprises 9% by weight of active ingredient).

IV. 20 parts by weight of a compound according to the invention are dissolved in a mixture composed of 60 parts by weight of cyclohexanone, 30 parts by weight of isobutanol, 5 parts by weight of the adduct of 7 mol of ethylene oxide and 1 mol of isooctylphenol and 5 parts by weight of the adduct of 40 mol of ethylene oxide and 1 mol of castor oil (comprises 16% by weight of active ingredient).

V. 80 parts by weight of a compound according to the invention are mixed thoroughly with 3 parts by weight of sodium diisobutylnaphthalene-alpha-sulfonate, 10 parts by weight of the sodium salt of a lignosulfonic acid from a sulfite waste liquor and 7 parts by weight of pulverulent silica gel, and the mixture is ground in a hammer mill (comprises 80% by weight of active ingredient).

VI. 90 parts by weight of a compound according to the invention are mixed with 10 parts by weight of N-methyl-α-pyrrolidone, which gives a solution which is suitable for use in the form of microdrops (comprises 90% by weight of active ingredient).

VII. 20 parts by weight of a compound according to the invention are dissolved in a mixture composed of 40 parts by weight of cyclohexanone, 30 parts by weight of isobutanol, 20 parts by weight of the adduct of 7 mol of ethylene oxide and 1 mol of isooctylphenol and 10 parts by weight of the adduct of 40 mol of ethylene oxide and 1 mol of castor oil. Pouring the solution into 100,000 parts by weight of water and finely distributing it therein gives an aqueous dispersion which comprises 0.02% by weight of the active ingredient.

VIII. 20 parts by weight of a compound according to the invention are mixed thoroughly with 3 parts by weight of sodium diisobutylnaphthalene-α-sulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active ingredient.

The active ingredients can be used as such, in the form of their formulations or the use forms prepared therefrom, e.g. in the form of directly sprayable solutions, powders, suspensions or dispersions, emulsions, oil dispersions, pastes, dusts, materials for spreading, or granules, by means of spraying, atomizing, dusting, scattering or pouring. The use forms depend entirely on the intended purposes; in any case, this is intended to guarantee the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances as such or dissolved in an oil or solvent, can be homogenized in water by means of wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within substantial ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even the active ingredient without additives.

Various types of oils, herbicides, fungicides, other pesticides, or bactericides may be added to the active ingredients, if appropriate also only immediately prior to use (tank mix). These agents can be admixed with the agents according to the invention in a weight ratio of 1:10 to 10:1.

In the use form as fungicides, the compositions according to the invention can also be present together with other active ingredients, e.g. with herbicides, insecticides, growth regulators, fungicides or else with fertilizers. Mixing the compounds I or the compositions comprising-them in the use form as fungicides with other fungicides frequently results in a broader fungicidal spectrum of action.

The following list of fungicides, together with which the compounds according to the invention can be used, is intended to illustrate the possible combinations, but not to impose any limitation:

sulfur, dithiocarbamates and their derivatives, such as iron (III) dimethyldithiocarbamate, zinc dimethyldithiocarbamate, zinc ethylenebisdithiocarbamate, manganese ethylenebisdithiocarbamate, manganese zinc ethylenediaminebisdithiocarbamate, tetramethylthiuram disulfide, ammonia complex of zinc (N,N-ethylenebisdithiocarbamate), ammonia complex of zinc (N,N′-propylenebisdithiocarbamate), zinc (N,N′-propylenebisdithiocarbamate), N,N′-polypropylenebis(thiocarbamoyl)disulfide;

nitro derivatives, such as dinitro(1-methylheptyl)phenyl crotonate, 2-sec-butyl-4,6-dinitrophenyl 3,3-dimethylacrylate, 2-sec-butyl-4,6-dinitrophenylisopropyl carbonate, diisopropyl 5-nitro-isophthalate;

heterocyclic substances, such as 2-heptadecyl-2-imidazoline acetate, 2,4-dichloro-6-(o-chloroanilino)-s-triazine, O,O-diethyl phthalimidophosphonothioate, 5-amino-1-[bis(dimethylamino)phosphinyl]-3-phenyl-1,2,4-triazole, 2,3-dicyano-1,4-dithioanthraquinone, 2-thio-1,3-dithiolo[4,5-b]quinoxaline, methyl 1-(butylcarbamoyl)-2-benzimidazolecarbamate, 2-methoxycarbonylaminobenzimidazole, 2-(2-furyl)benzimidazole, 2-(4-thiazolyl)benzimidazole, N-(1,1,2,2-tetrachloroethylthio)tetrahydrophthalimide, N-trichloromethylthiotetrahydrophthalimide, N-trichloromethylthiophthalimide, 5-Chloro-2-cyano-4-p-tolyl-imidazole-1-sulfonic acid dimethylamide, N-dichlorofluoromethylthio-N′,N′-dimethyl-N-phenylsulfo-diamide, 5-ethoxy-3-trichloromethyl-1,2,3-thiadiazole, 2-thiocyanatomethylthiobenzothiazole, 1,4-dichloro-2,5-dimethoxybenzene, 4-(2-chlorophenylhydrazono)-3-methyl-5-isoxazolone, pyridine-2-thiol1-oxide, 8-hydroxyquinoline or its copper salt, 2,3-dihydro-5-carboxanilido-6-methyl-1,4-oxathiine, 2,3-dihydro-5-carboxanilido-6-methyl-1,4-oxathiine 4,4-dioxide, 2-methyl-5,6-dihydro-4H-pyran-3-carboxanilide, 2-methylfuran-3-carboxanilide, 2,5-dimethylfuran-3-carboxanilide, 2-Chloro-N-(4′-chloro-biphenyl-2-yl)-nicotinamide, 2,4,5-trimethylfuran-3-carboxanilide, N-cyclohexyl-2,5-dimethylfuran-3-carboxamide, N-cyclohexyl-N-methoxy-2,5-dimethylfuran-3-carboxamide, 2-methylbenzanilide, 2-iodobenzanilide, N-formyl-N-morpholine-2,2,2-trichloroethyl acetal, piperazine-1,4-diylbis-1-(2,2,2-trichloroethyl)formamide, 1-(3,4-dichloroanilino)-1-formylamino-2,2,2-trichloroethane; 2,6-dimethyl-N-tridecylmorpholine or its salts, 2,6-dimethyl-N-cyclododecylmorpholine or its salts, N-[3-(p-tert-butylphenyl)-2-methylpropyl]-cis-2,6-dimethyl-morpholine, N-[3-(p-tert-butylphenyl)-2-methylpropyl]-piperidine, 1-[2-(2,4-dichlorophenyl)-4-ethyl-1,3-dioxolan-2-yl-ethyl]-1H-1,2,4-triazole, 1-[2-(2,4-dichlorophenyl)-4-n-propyl-1,3-dioxolan-2-yl-ethyl]-1H-1,2,4-triazole, N-(n-propyl)-N-(2,4,6-trichlorophenoxyethyl)-N′-imidazolyl-urea, 1-(4-chlorophenoxy)-3,3-dimethyl-1-(1H-1,2,4-triazol-1-yl)-2-butanone, 1-(4-chlorophenoxy)-3,3-dimethyl-1-(1H-1,2,4-triazol-1-yl)-2-butanol, (2RS,3RS)-1-[3-(2-chlorophenyl)-2-(4-fluorophenyl)-oxiran-2-ylmethyl]-1H-1,2,4-triazole, α-(2-chlorophenyl)-α-(4-chlorophenyl)-5-pyrimidinemethanol, 5-butyl-2-dimethylamino-4-hydroxy-6-methylpyrimidine, bis(p-chlorophenyl)-3-pyridinemethanol, 1,2-bis(3-ethoxycarbonyl-2-thioureido)benzene, 1,2-bis(3-methoxycarbonyl-2-thioureido)benzene,

strobilurines such as azoxystrobin, kresoxim methyl, methyl-E-methoxyimino-[α-(2-phenoxyphenyl)]-acetamide, methyl E-methoxyimino-[(α-(2,5-dimethylphenoxy)-o-tolyl]acetamide, picoxystrobin, pyraclostrobin, trifloxystrobin, anilinopyrimidines such as N-(4,6-dimethylpyrimidin-2-yl)aniline, N-[4-methyl-6-(1-propynyl)pyrimidin-2-yl]-aniline, N-[4-methyl-6-cyclopropylpyrimidin-2-yl]aniline, phenylpyrroles such as 4-(2,2-difluoro-1,3-benzodioxol-4-yl)pyrrole-3-carbonitrile,

cinnamamides such as 3-(4-chlorophenyl)-3-(3,4-dimethoxyphenyl)acryloylmorpholine, 3-(4-fluorophenyl)-3-(3,4-dimethoxy-phenyl)acryloylmorpholine,

and a variety of fungicides such as dodecylguanidine acetate, 3-[3-(3,5-dimethyl-2-oxycyclohexyl)-2-hydroxyethyl]glutarimide, hexachlorobenzene, methyl N-(2,6-dimethylphenyl)-N-(2-furoyl)-DL-alaninate, DL-N-(2,6-dimethylphenyl)-N-(2′-methoxyacetyl)-alanine methyl ester, N-(2,6-dimethylphenyl)-N-chloroacetyl-D,L-2-amino-butyrolactone, DL-N-(2,6-dimethylphenyl)-N-(phenylacetyl)alanine methyl ester, 5-methyl-5-vinyl-3-(3,5-dichlorophenyl)-2,4-dioxo-1,3-oxazolidine, 3-[3,5-dichlorophenyl(5-methyl-5-methoxymethyl]-1,3-oxazolidine-2,4-dione, 3-(3,5-dichlorophenyl)-1-isopropylcarbamoylhydantoin, N-(3,5-dichlorophenyl)-1,2-dimethylcyclopropane-1,2-dicarboximide, 2-cyano-[N-(ethylaminocarbonyl)-2-methoximino]acetamide, 3,5-Dichloro-N-(3-chloro-1-ethyl-1-methyl-2-oxo-propyl)-4-methyl-benzamide, 1-(3-Bromo-6-methoxy-2-methyl-phenyl)-1-(2,3,4-trimethoxy-6-methyl-phenyl)-methanone, 1-[2-(2,4-dichloro-phenyl)pentyl]-1H-1,2,4-triazole, 2,4-difluoro-α-(1H-1,2,4-triazolyl-1-methyl)benzhydryl alcohol, N-(3-chloro-2,6-dinitro-4-trifluoromethylphenyl)-5-trifluoromethyl-3-chloro-2-aminopyridine, 1-((bis(4-fluorophenyl)methylsilyl)methyl)-1H-1,2,4-triazole.

### SYNTHESIS EXAMPLES

With due modification of the starting compounds, the protocols shown in the synthesis examples below were used for obtaining further compounds I. The resulting compounds I, together with physical data, are listed in Table I which follows.

### Example 1

### Preparation of diethyl (2-fluoro-6-methylphenyl)-malonate

Diethyl malonate (0.49 mol) was added to a mixture of sodium hydride (0.51 mol) and 1,4-dioxane (140 ml) at 60° C. within 2 hours. The mixture was stirred for 10 minutes at 60° C. and copper (I) bromide (0.05 mol) was added. After 15 minutes, a solution of 2-bromo-3-fluorotoluene (0.25 mol) in 10 ml 1,4-dioxane was added. The reaction mixture was kept at 100° C. for about 15 hours and after cooling to about 15° C. 35 ml of 12N Hydrochloric acid were added. The precipitate was filtered off, the filtrate was extracted with diethyl ether. The organic phase was separated, dried and filtered. The filtrate was evaporated to yield 42 g of the title compound.

### Example 2

### Preparation of 5,7-dihydroxy-6-(2-fluoro-6-methylphenyl)-[1,2,4]-triazolo-[1,5-α]pyrimidine

A mixture of 3-amino-1,2,4-triazole (14 g), diethyl (2-fluoro-6-methylphenyl)-malonate (0.17 mol, Ex. 1) and tributylamine (50 ml) is heated at 180° C. for six hours; after coooling to 70° C. a solution of 21 g sodium hydroxide in 200 ml water was added and the reaction mixture was stirred for 30 minutes. The organic phase was separated off and the aqueous phase was extracted with diethyl ether. The aqueous phase was acidified with concentrated hydrochloric acid. The precipitate was collected by filtration and dried to yield 41 g of the title compound.

### Example 3

### Preparation of 5,7-dichloro-6-(2-fluoro-6-methylphenyl)-[1,2,4]-triazolo-[1,5-α]pyrimidine

A mixture of the compound from Ex. 2 (30 g) and phosphorous oxychloride (50 ml) was refluxed for eight hours. Phosphorous oxychloride was partly distilled off. The residue was poured into a mixture of dichloromethane and water. The organic layer was separated, dried and filtered. The filtrate was concentrated in vacuo to yield 27 g of the title compound of mp. 130° C.

### Example 4

### Preparation of 5-chloro-6-(2-fluoro-6-methylphenyl)-7-isopropylamino-[1,2,4]-triazolo[1,5-α]pyrimidine [I-8]

A mixture of isopropylamine (1.5 mmol), triethylamine (1.5 mmol) and dichloromethane (10 ml) was added to a solution of 1.5 mmol of the product from Ex. 3 in 20 ml dichloromethane under stirring. The reaction mixture was stirred for about 16 hours at about 20 to 25° C. and subsequently washed with 5% hydrocloric acid. The organic layer was separated, dried and filtered. The filtrate was evaporated and the residue was chromatographed to yield 0.46 g of the title compound of mp. 128° C.

### Example 5

### Preparation of 5-cyano-6-(2-fluoro-6-methylphenyl)-7-(4-methylpiperidin-1-yl)-[1,2,4]-triazolo[1,5-α]pyrimidine [I-49]

A mixture of 0.1 mol compound I-9 and tetraethylammonium cyanide (0.25 mol) in 750 ml Dimethylformamide (DMF) was stirred for 16 hours at about 20 to 25° C. To this mixture was added water and methyl-tert.butyl ether (MTBE), the organic phase was separated, washed with water, dried and filtered. The filtrate was evaporated and the residue was chromatographed to yield 6.51 g of the title compound of mp. 211° C.

### Example 6

### Preparation of 5-methoxy-6-(2-fluoro-6-methylphenyl)-7-(4-methylpiperidin-1-yl)-[1,2,4]-triazolo[1,5-α]pyrimidine [I-50]

After having added a solution of sodium methanolate (30%, 71.5 mmol) to a solution of 65 mmol of compound I-9 in 400 ml dry methanol was added this mixture was stirred for about 16 hours at about 20 to 25° C. Methanol was evaporated and the residue was dissolved with dichloromethane. The organic phase was washed with water, dried and filtered. The filtrate was evaporated and the residue was chromatographed to yield 4.32 g of the title compound of mp. 142° C.

TABLE I

| | |
|---|---|
| | I |
| 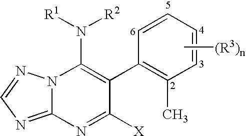 | |

| | | | | | phys. data |
|---|---|---|---|---|---|
| No. | R¹ | R² | (R³)ₙ | X | (m.p.[° C.]; ¹H-NMR δ [ppm]) |
| I-1 | CH₂C(CH₃)═CH₂ | CH₂CH₃ | 4,6-(CH₃)₂ | Cl | 131 |
| I-2 | —(CH₂)₂CH(CH₃)(CH₂)₂— | | 4,6-(CH₃)₂ | Cl | 138 |
| I-3 | CH₂C(CH₃)═CH₂ | CH₂CH₃ | 4-OCH₃-6-CH₃ | Cl | 74 |
| I-4 | (±) CH(CH₃)—CH(CH₃)₃ | H | 4-OCH₃-6-CH₃ | Cl | 163 |
| I-5 | CH₂CF₃ | H | 4-OCH₃-6-CH₃ | Cl | 191 |
| I-6 | H | H | 6-F | Cl | 250 |
| I-7 | CH₂C(CH₃)═CH₂ | CH₂CH₃ | 6-F | Cl | 124 |
| I-8 | CH(CH₃)₂ | H | 6-F | Cl | 128 |
| I-9 | —(CH₂)₂CH(CH₃)(CH₂)₂— | | 6-F | Cl | 142 |
| I-10 | cyclopentyl | H | 6-F | Cl | 159 |
| I-11 | CH₂CH₃ | CH₂CH₃ | 6-F | Cl | 147 |
| I-12 | CH₂CH₂CH₃ | CH₂CH₂CH₃ | 6-F | Cl | 124 |
| I-13 | CH(CH₃)₂ | CH₃ | 6-F | Cl | 159 |
| I-14 | (±) CH(CH₃)—CH₂CH₃ | H | 6-F | Cl | 115 |
| I-15 | (S) CH(CH₃)—CH₂CH₃ | H | 6-F | Cl | 123 |
| I-16 | (R) CH(CH₃)—CH₂CH₃ | H | 6-F | Cl | 129 |
| I-17 | (±) CH(CH₃)—CH(CH₃)₂ | H | 6-F | Cl | A) 133/B) 138 |
| I-18 | (S) CH(CH₃)—CH(CH₃)₂ | H | 6-F | Cl | A) 120/B) 130 |
| I-19 | (R) CH(CH₃)—CH(CH₃)₂ | H | 6-F | Cl | A) 119/B) 131 |
| I-20 | (±) CH(CH₃)—CH(CH₃)₃ | H | 6-F | Cl | A) 148/B) 174 |
| I-21 | (S) CH(CH₃)—CH(CH₃)₃ | H | 6-F | Cl | A) 160/B) 203 |
| I-22 | (R) CH(CH₃)—CH(CH₃)₃ | H | 6-F | Cl | A) 159/B) 203 |
| I-23 | (±) CH(CH₃)—CF₃ | H | 6-F | Cl | A) 149/B) 56 |
| I-24 | (S) CH(CH₃)—CF₃ | H | 6-F | Cl | A) 166/B) 70 |
| I-25 | (R) CH(CH₃)—CF₃ | H | 6-F | Cl | A) 167/B) 70 |
| I-26 | CH₂CF₃ | H | 6-F | Cl | 173 |
| I-27 | H | H | 4-F | Cl | 281 |
| I-28 | CH₂C(CH₃)═CH₂ | CH₂CH₃ | 4-F | Cl | 115 |
| I-29 | CH(CH₃)₂ | H | 4-F | Cl | 94 |
| I-30 | —(CH₂)₂CH(CH₃)(CH₂)₂— | | 4-F | Cl | 168 |
| I-31 | cyclopentyl | H | 4-F | Cl | 141 |
| I-32 | CH₂CH₃ | CH₂CH₃ | 4-F | Cl | 156 |
| I-33 | CH₂CH₂CH₃ | CH₂CH₂CH₃ | 4-F | Cl | 121 |
| I-34 | CH(CH₃)₂ | CH₃ | 4-F | Cl | 153 |
| I-35 | (±) CH(CH₃)—CH₂CH₃ | H | 4-F | Cl | 118 |
| I-36 | (S) CH(CH₃)—CH₂CH₃ | H | 4-F | Cl | 125 |
| I-37 | (R) CH(CH₃)—CH₂CH₃ | H | 4-F | Cl | 126 |
| I-38 | (±) CH(CH₃)—CH(CH₃)₂ | H | 4-F | Cl | 132 |
| I-39 | (S) CH(CH₃)—CH(CH₃)₂ | H | 4-F | Cl | 124 |
| I-40 | (R) CH(CH₃)—CH(CH₃)₂ | H | 4-F | Cl | 124 |
| I-41 | (±) CH(CH₃)—CH(CH₃)₃ | H | 4-F | Cl | 162 |
| I-42 | (S) CH(CH₃)—CH(CH₃)₃ | H | 4-F | Cl | 156 |
| I-43 | (R) CH(CH₃)—CH(CH₃)₃ | H | 4-F | Cl | 156 |
| I-44 | (±) CH(CH₃)—CF₃ | H | 4-F | Cl | 163 |
| I-45 | (S) CH(CH₃)—CF₃ | H | 4-F | Cl | 145 |
| I-46 | (R) CH(CH₃)—CF₃ | H | 4-F | Cl | 145 |
| I-47 | CH₂CF₃ | H | 4-F | Cl | 167 |
| I-48 | CH₂CF₃ | H | 4-F-6-CH₃ | Cl | 220 |
| I-49 | —(CH₂)₂CH(CH₃)(CH₂)₂— | | 6-F | CN | 211 |
| I-50 | —(CH₂)₂CH(CH₃)(CH₂)₂— | | 6-F | OCH₃ | 142 |
| I-51 | —(CH₂)₂CH(CH₃)(CH₂)₂— | | 6-F | CH₃ | 145 |
| I-52 | cyclopentyl | H | 4,6-(CH₃)₂ | Cl | 200 |
| I-53 | CH₂CH₃ | CH₂CH₃ | 4,6-(CH₃)₂ | Cl | 105 |
| I-54 | CH₂CH₂CH₃ | CH₂CH₂CH₃ | 4,6-(CH₃)₂ | Cl | 99 |
| I-55 | (±) CH(CH₃)—CH₂CH₃ | H | 4,6-(CH₃)₂ | Cl | 191 |
| I-56 | (S) CH(CH₃)—CH₂CH₃ | H | 4,6-(CH₃)₂ | Cl | 184 |
| I-57 | (R) CH(CH₃)—CH₂CH₃ | H | 4,6-(CH₃)₂ | Cl | 184 |
| I-58 | (±) CH(CH₃)—CH(CH₃)₂ | H | 4,6-(CH₃)₂ | Cl | 107 |
| I-59 | (S) CH(CH₃)—CH(CH₃)₂ | H | 4,6-(CH₃)₂ | Cl | 87 |
| I-60 | (R) CH(CH₃)—CH(CH₃)₂ | H | 4,6-(CH₃)₂ | Cl | 87 |
| I-61 | (±) CH(CH₃)—CH(CH₃)₃ | H | 4,6-(CH₃)₂ | Cl | 79 |
| I-62 | (S) CH(CH₃)—CH(CH₃)₃ | H | 4,6-(CH₃)₂ | Cl | 81 |
| I-63 | (R) CH(CH₃)—CH(CH₃)₃ | H | 4,6-(CH₃)₂ | Cl | 81 |
| I-64 | CH₂C(CH₃)═CH₂ | CH₂CH₃ | 4-Cl | Cl | 137 |
| I-65 | CH(CH₃)₂ | H | 4-Cl | Cl | 136 |
| I-66 | —(CH₂)₂CH(CH₃)(CH₂)₂— | | 4-Cl | Cl | 173 |
| I-67 | cyclopentyl | H | 4-Cl | Cl | 147 |
| I-68 | CH₂CH₃ | CH₂CH₃ | 4-Cl | Cl | 167 |
| I-69 | CH₂CH₂CH₃ | CH₂CH₂CH₃ | 4-Cl | Cl | 149 |
| I-70 | CH(CH₃)₂ | CH₃ | 4-Cl | Cl | 159 |
| I-71 | (±) CH(CH₃)—CH₂CH₃ | H | 4-Cl | Cl | 128 |
| I-72 | (S) CH(CH₃)—CH₂CH₃ | H | 4-Cl | Cl | 114 |
| I-73 | (R) CH(CH₃)—CH₂CH₃ | H | 4-Cl | Cl | 114 |
| I-74 | (±) CH(CH₃)—CH(CH₃)₂ | H | 4-Cl | Cl | 116 |
| I-75 | (S) CH(CH₃)—CH(CH₃)₂ | H | 4-Cl | Cl | 130 |
| I-76 | (R) CH(CH₃)—CH(CH₃)₂ | H | 4-Cl | Cl | 130 |
| I-77 | (±) CH(CH₃)—CH(CH₃)₃ | H | 4-Cl | Cl | 159 |
| I-78 | (S) CH(CH₃)—CH(CH₃)₃ | H | 4-Cl | Cl | 159 |
| I-79 | (R) CH(CH₃)—CH(CH₃)₃ | H | 4-Cl | Cl | 159 |
| I-80 | (±) CH(CH₃)—CF₃ | H | 4-Cl | Cl | 184 |
| I-81 | (S) CH(CH₃)—CF₃ | H | 4-Cl | Cl | 143 |
| I-82 | (R) CH(CH₃)—CF₃ | H | 4-Cl | Cl | 143 |
| I-83 | CH₂CF₃ | H | 4-Cl | Cl | 206 |
| I-84 | CH₂C(CH₃)═CH₂ | CH₂CH₃ | 4-CH₃ | Cl | 158 |
| I-85 | CH(CH₃)₂ | H | 4-CH₃ | Cl | 145 |
| I-86 | CH₂CH₃ | CH₂CH₃ | 4-CH₃ | Cl | 141 |
| I-87 | (±) CH(CH₃)—CH₂CH₃ | H | 4-CH₃ | Cl | 122 |
| I-88 | (S) CH(CH₃)—CH(CH₃)₃ | H | 4-CH₃ | Cl | 108 |
| I-89 | (R) CH(CH₃)—CH(CH₃)₃ | H | 4-CH₃ | Cl | 108 |
| I-90 | —(CH₂)₂CH(CH₃)(CH₂)₂— | | 4-COOCH₃ | Cl | 8.45(s); 8.05(s); 7.95(d); |
| | | | | | 7.25(d); 4.0(s); 3.85(d); 3.45(d); |
| | | | | | 2.7(t); 2.65(t); 2.25(s); 1.55(m); |
| | | | | | 1.45(m); 1.25(m); 0.9(d) |
| I-91 | (R) CH(CH₃)—CH(CH₃)₂ | H | 4-COOCH₃ | Cl | 8.35(s); 8.1(s); 8.0(d); 7.35(d); |
| | | | | | 6.2(m); 3.95(s); 3.1(m); 2.25(s); |
| | | | | | 1.55(sept); 1.2(d); 0.95(d); |
| | | | | | 0.9(d) |
| I-92 | (S) CH(CH₃)—CF₃ | H | 4-COOCH₃ | Cl | 8.4(s); 8.15(s); 8.05(d); 7.35(m); |
| | | | | | 5.7(m); 4.6(m); 3.95(s); 2.3(s); |
| | | | | | 2.25(s); 1.4(d); 1.3(d) |
| I-93 | CH₂CF₂CF₃ | H | 6-F | Cl | 162 |
| I-94 | CH₂CF₂CF₂CF₃ | H | 6-F | Cl | 165 |
| I-95 | CH₂CF₂CF₃ | H | 4-F | Cl | 176 |
| I-96 | CH₂CF₂CF₂CF₃ | H | 4-F | Cl | 138 |
| I-97 | CH₂CF₂CF₃ | H | 4-Cl | Cl | 164 |
| I-98 | CH₂CF₂CF₂CF₃ | H | 4-Cl | Cl | 126 |
| I-99 | H | H | 4-Cl | Cl | 276 |

In some cases of chiral groups R¹ and due to the hindered rotation of the phenyl group two diastereomers A) and B) exist which may differ in their physical properties.

### Examples of the Action Against Harmful Fungi

The fungicidal action of the compounds of the formula I was demonstrated by the following experiments:

The active compounds, separately or together, were formulated as a 10% emulsion in a mixture of 70% by weight of cyclohexanone, 20% by weight of Nekanil® LN (Lutensol® AP6, wetting agent having emulsifying and dispersant action based on ethoxylated alkylphenols) and 10% by weight of Wettol® EM (nonionic emulsifier based on ethoxylated castor oil) and diluted with water to the desired concentration.

Compound A known from WO-A 98/46608 (No. 9) served as comparative active compound:

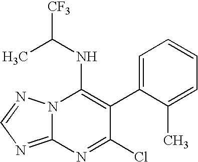

Comparison Trial 1—Fungicidal Control of Early Blight on Tomatoes (Alternaria solani)

Young seedlings of tomato plants of the variety “Groβe Fleischtomate St. Pierre” were grown in pots to the 2 to 4 leaf stage. These plants were sprayed to run-off with an aqueous suspension, containing the concentration of active ingredient mentioned in the table below, prepared from a stock solution containing 10% of the active ingredient, 85% cyclohexanone and 5% emulsifier. The next day, the treated plants were inoculated with an aqueous suspension of Alternaria solani containing 0.2×10⁶ spores per ml. Then the trial plants were immediately transferred to a humid chamber. After 6 days at 20 to 23° C. and a relative humidity close to 100%, the extent of fungal attack on the leaves was visually assessed as % diseased leaf area.

In this test, the plants which had been treated with 63 ppm of compounds I-23 and I-24, resp., showed an infection of not more than 3%, whereas the the plants treated with 63 ppm of the comparison compound A were infected to 15%, and the unteated plants were infected to 90%.

Comparison Trial 2—Fungicidal Control of Powdery Mildew on Wheat Caused by Blumeria graminis f. sp. tritici

The first fully developed leaves of pot grown wheat of the variety “Kanzler” were sprayed to run-off with an aqueous suspension, containing the concentration of active ingredient or their mixture mentioned in the table below, prepared from a stock solution containing 10% of the active ingredient, 85% cyclohexanone and 5% emulsifier. The next day the treated plants were inoculated with spores of Blumeria graminis f. sp. tritici by shaking heavily infestated stock plants over the treated pots. After cultivation in the greenhouse for 7 days at 22-26° C. and a relative humidity between 60 to 90% the extent of fungal attack on the leaves was visually assessed as % diseased leaf area.

In this test, the plants which had been treated with 63 ppm of compounds I-23 and I-24, resp., showed an infection of not more than 3%, whereas the the plants treated with 63 ppm of the comparison compound A were infected to 30%, and the unteated plants were infected to 85%.

Comparison Trial 3—Fungicidal Control of Grape Downy Mildew (Plasmopara viticola)

Grape cuttings of the cultivar “Müller-Thurgau” were grown in pots to the 4 to 5 leaf stage. These plants were sprayed to run-off with an aqueous suspension, containing the concentration of active ingredient or their mixture mentioned in the table below, prepared from a stock solution containing 10% of the active ingredient, 85% cyclohexanone and 5% emulsifier. The plants were allowed to air-dry. The next day they were inoculated with an aqueous spore suspension of Plasmopara viticola by spraying it at the lower leaf-side. Then the trial plants were immediately transferred for 24 h to a humid chamber with 22-24° C. and a relative humidity close to 100%. For a period of 5 days, cultivation followed in a greenhouse at 20-25° C. and a relative humidity about 50-80%. To stimulate the outbreak of the disease symptoms, the plants were transferred to a humid chamber again for 24 hours. Then the extent of fungal attack on the lower leaf surface was visually assessed as % diseased leaf area.

In this test, the plants which had been treated with 250 ppm of compounds I-23 and I-24, resp., showed an infection of not more than 7%, whereas the the plants treated with 250 ppm of the comparison compound A, and the unteated plants were infected to 80%.

Use Example 1—Fungicidal Control of Early Blight on Tomatoes (Alternaria solani)

The test was conducted as described in Comparison trial 1.

In this test, the plants which have been treated with 250 ppm of compounds I-7, I-9, I-10, I-28, I-30, I-35, I-38, I-41, I-44, I-66, and I-83, resp., showed no infection, whereas the untreated plants were infected to 90%.

Use Example 2—Fungicidal Control of Powdery Mildew on Wheat Caused by Blumeria graminis f. sp. tritici

The test was conducted as described in Comparison trial 2.

In this test, the plants which have been treated with 250 ppm of compounds I-14, I-28, I-30, I-35, I-38, I-41, and I-44, resp., showed an infection of not more than 5%, whereas the untreated plants were infected to 85%.

Use Example 3—Fungicidal Control of Grape Downy Mildew (Plasmopara viticola)

Leaves of potted vines of the “Müller Thurgau” variety were sprayed with aqueous liquors made from a stock solution consisting of 10% of active ingredient, 85% of cyclohexanone, and 5% of emulsifier. To assess the duration of action, the plants were set up, after the sprayed-on layer had dried, in the greenhouse for 8 days. Then the leaves were infected with a zoospore suspension of the fungus Plasmopara viticola, first placed in a vapoursaturated chamber at 24° C., and then kept for 5 days in a greenhouse at 20 to 30° C. To accelerate and intensify the sporangiophore discharge, the plants were then again placed in the moist chamber for 16 hours. The extent of fungus attack was then assessed on the undersides of the leaves.

In this test, the plants which have been treated with 250 ppm of compounds I-7, I-9, I-10, I-35, I-38, and I-44, resp., showed an infection of 3 to 40%, whereas the untreated plants were infected to 80%.

1. Substituted 6-(2-tolyl)-triazolopyrimidines of formula I 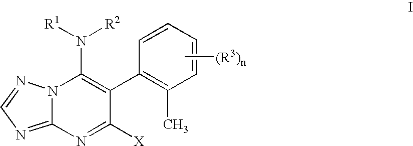
in which
R¹ and R² independently denote hydrogen or
C₁-C₁₀-alkyl, C₂-C₁₀-alkenyl, C₂-C₁₀-alkynyl, or C₄-C₁₀-alkadienyl, C₁-C₁₀-haloalkyl, C₂-C₁₀-haloalkenyl, C₃-C₁₀-cycloalkyl, phenyl, naphthyl, or
C₃-C₁₀-cycloalkyl, phenyl, naphthyl, or
5- or 6-membered heterocyclyl, containing one to four nitrogen atoms or one to three nitrogen atoms and one sulfur or oxygen atom, or
5- or 6-membered heteroaryl, containing one to four nitrogen atoms or one to three nitrogen atoms and one sulfur or oxygen atom, or
where R¹ and R² radicals may be unsubstituted or partly or fully halogenated or may carry one to three groups R^{a},
R^{a} is cyano, nitro, hydroxyl, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and C₁-C₄-alkylenedioxy; or
R¹ and R² together with the interjacent nitrogen atom represent a 5- or 6-membered heterocyclic ring, containing one to four nitrogen atoms or one to three nitrogen atoms and one sulfur or oxygen atom, which may be substituted by one to three R^{a} radicals;
R³ is halogen, cyano, C₁-C₁₀-alkyl, C₁-C₁₀-alkoxy, C₁-C₁₀-haloalkyl, or C(═O)A, wherein
A is hydrogen, hydroxy, C₁-C₈-alkyl, C₁-C₈-alkoxy, amino, C₁-C₈-alkylamino, or di-(C₁-C₈-alkyl)amino;
n is an integer from 1 to 4; and
X is halogen, cyano, C₁-C₆-alkyl, C₁-C₆-alkoxy, C₁-C₆-haloalkoxy or C₃-C₈-alkenyloxy.

2. Compounds of formula I according to claim 1, in which
R¹ is straight chained or branched C₁-C₆-alkyl, C₂-C₆-alkenyl, C₃-C₉-cycloalkyl, or C₁-C₁₀-haloalkyl, and
R² is hydrogen or C₁-C₆-alkyl, or
R¹ and R² together with the interjacent nitrogen atom represent a heterocyclic ring with 5 or 6 carbon atoms being optionally substituted with one or two C₁-C₆-alkyl groups.

3. Compounds of formula I according to claims 1 or 2 in which R² is hydrogen.

4. Compounds of formula I according to claims 1 to 3 in which X is halogen.

5. Compounds of formula I according to claims 1 to 4 in which (R³)ₙ is 4-(C₁-C₈-alkoxy-carbonyl) or 4-cyano.

6. A process for the preparation of compounds of formula I as defined in claim 4 which comprises reacting 5-amino-1,2,4-triazole 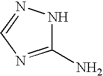
with 2-phenyl-substituted malonic acid ester of formula II, 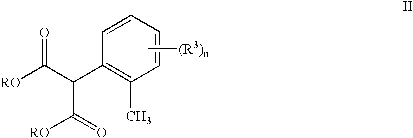
wherein R³ and n are as defined in formula I, and R denotes C₁-C₆-alkyl, under alkaline conditions, to yield compounds of formula III, 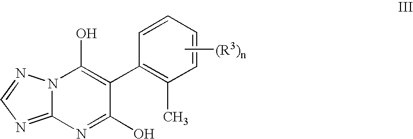
which are subsequently treated with a halogenating agent to give 5,7-dihalogen-6-phenyl-triazolopyrimidines of formula IV 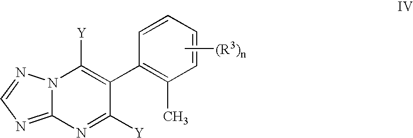
in which Y is halogen with an amine of formula V 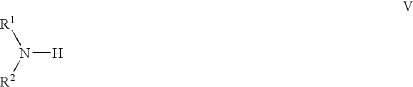
in which R¹ and R² are as defined in formula I to produce compounds of formula I.

7. A process for the preparation of compounds of formula I according to claim 1 wherein X is cyano, C₁-C₁₀-alkoxy, or C₁-C₁₀-haloalkyl, which comprises reacting 5-halogen-triazolopyrimidine of formula I 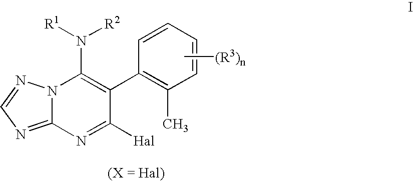
with compounds of formula VI,
M—X′ VI
which are, dependent from the value of X′ to be introduced, an anorganic cyano salt, an alkoxylate, haloalkoxylate or an alkenyloxylate, resp., wherein M is ammonium-, tetraalkylammonium-, alkalimetal- or earth metal cation, to produce compounds of formula I.

8. Intermediates of formulae II, III, and IV as defined in claim 6.

9. A composition suitable for controlling phytopathogenic fungi, comprising a solid or liquid carrier and a compound of the formula I as claimed in claim 1.

10. A method for controlling phytopathogenic fungi, which comprises treating the fungi or the materials, plants, the soil or the seed to be protected against fungal attack with an effective amount of a compound of the formula I as claimed in claim 1.
